SEQUENCE LISTING (2 sequences)

SEQ ID NO: 1 - DNA, 2867 bp, Homo sapiens
cgaggtggtgaggagagctggttgcgtgagtctcctcagctctgcttaccggtgcgactagcggcagcgacgcggctaaaagcgaaggggcgagtgcgagtcccctgagctgtacgaacgcggtcgccatggaccgcccagatgaggggcctccggccaagacccgccgcctgagcagctccgagtctccacagcgcgacccgcccccgccgccgccgccgccgccgctcctccgactgccgctgcctccaccccagcagcgcccgaggctccaggaggaaacggaggcggcacaggtgctggccgatatgaggggggtgggactgggccccgcgctgcccccgccgcctccctatgtcattctcgaggagggggggatccgcgcatacttcacgctcggtgctgagtgtcccggctgggattctaccatcgagtcggggtatggggaggcgcccccgcccacggagagcctggaagcactccccactcctgaggcctcgggggggagcctggaaatcgattttcaggttgtacagtcgagcagttttggtggagagggggccctagaaacctgtagcgcagtggggtgggcgccccagaggttagttgacccgaagagcaaggaagaggcgatcatcatagtggaggatgaggatgaggatgagcgggagagtatgaggagcagcaggaggcggcggcggcggcggaggaggaagcagaggaaggtgaagagggaaagcagagagagaaatgccgagaggatggagagcatcctgcaggcactggaggatattcagctggatctggaggcagtgaacatcaaggcaggcaaagccttcctgcgtctcaagcgcaagttcatccagatgcgaagacccttcctggagcgcagagacctcatcatccagcatatcccaggcttctgggtcaaagcattcctcaaccaccccagaatttcaattttgatcaaccgacgtgatgaagacattttccgctacttgaccaatctgcaggtacaggatctcagacatatctccatgggctacaaaatgaagctgtacttccagactaacccctacttcacaaacatggtgattgtcaaggagttccagcgcaaccgctcaggccggctggtgtctcactcaaccccaatccgctggcaccggggccaggaaccccaggcccgtcgtcacgggaaccaggatgcgagccacagctttttcagctggttctcaaaccatagcctcccagaggctgacaggattgctgagattatcaagaatgatctgtgggttaaccctctacgctactacctgagagaaaggggctccaggataaagagaaagaagcaagaaatgaagaaacgtaaaaccaggggcagatgtgaggtggtgatcatggaagacgcccctgactattatgcagtggaagacattttcagcgagatctcagacattgatgagacaattcatgacatcaagatctctgacttcatggagaccaccgactacttcgagaccactgacaatgagataactgacatcaatgagaacatctgcgacagcgagaatcctgaccacaatgaggtccccaacaacgagaccactgataacaacgagagcgctgatgaccacgaaaccactgacaacaatgagagtgcagatgacaacaacgagaatcctgaagacaataacaagaacactgatgacaacgaagagaaccctaacaacaacgagaacacttacggcaacaacttcttcaaaggtggcttctggggcagccatggcaacaaccaggacagcagcgacagtgacaatgaagcagatgaggccagtgatgatgaagataatgatggcaacgaaggtgacaatgagggcagtgatgatgatggcaatgaaggtgacaatgaaggcagcgatgatgacgacagagacattgagtactatgagaaagttattgaagactttgacaaggatcaggctgactacgaggacgtgatagagatcatctcagacgaatcagtggaagaagagggcattgaggaaggcatccagcaagatgaggacatctatgaggaaggaaactatgaggaggaaggaagtgaagatgtctgggaagaaggggaagattcggacgactctgacctagaggatgtgcttcaggtcccaaacggttgggccaatccggggaagagggggaaaaccggataagggttttccccttttggggatcacctctctgtatcccccacccactatcccatttgccctcctcctcagctagggccacgcggccccacattgcacttctggggggtgaccgacttcgtacacgggtttaaagtttatttttatggtttagtcattgcagagttcttattttggggggagggaaagggggctagtccccttcttttggccctccgcccccgcaggcttctgtgtgctgctaactgtatttattgtgatgccttggtcagggcccctctacccacttctcccagtcagttgtggccccagcccctctccctgtgctgtgtggagtggacaccctgacccccgaagcggggagggccgctgtggccttcgtcacagccgcgcagtgcccatggaggcgctgctgccaccttcctctcccaagttctttctccatccctctcctcttcccgccgcgccgctagcccgcctcggtgtctatgcaaggccgcttcgccattgcggtattctttgcggtattcttgtccccgtcccccagaaggctcgcctctccccgtggaccctgttaatcccaataaaattctgagcaagttcaaaaaaaaaaaaaaaaaaaaaaaaaaaaaaaaaaaaaaaaaaaaaaaaaaaaaaaaa

SEQ ID NO: 2 - protein, 693 aa, Homo sapiens
MDRPDEGPPAKTRRLSSSESPQRDPPPPPPPPPLLRLPLPPPQQRPRLQEETEAAQVLADMRGVGLGPALPPPPPYVILEEGGIRAYFTLGAECPGWDSTIESGYGEAPPPTESLEALPTPEASGGSLEIDFQVVQSSSFGGEGALETCSAVGWAPQRLVDPKSKEEAIIIVEDEDEDERESMRSSRRRRRRRRRKQRKVKRESRERNAERMESILQALEDIQLDLEAVNIKAGKAFLRLKRKFIQMRRPFLERRDLIIQHIPGFWVKAFLNHPRISILINRRDEDIFRYLTNLQVQDLRHISMGYKMKLYFQTNPYFTNMVIVKEFQRNRSGRLVSHSTPIRWHRGQEPQARRHGNQDASHSFFSWFSNHSLPEADRIAEIIKNDLWVNPLRYYLRERGSRIKRKKQEMKKRKTRGRCEVVIMEDAPDYYAVEDIFSEISDIDETIHDIKISDFMETTDYFETTDNEITDINENICDSENPDHNEVPNNETTDNNESADDHETTDNNESADDNNENPEDNNKNTDDNEENPNNNENTYGNNFFKGGFWGSHGNNQDSSDSDNEADEASDDEDNDGNEGDNEGSDDDGNEGDNEGSDDDDRDIEYYEKVIEDFDKDQADYEDVIEIISDESVEEEGIEEGIQQDEDIYEEGNYEEEGSEDVWEEGEDSDDSDLEDVLQVPNGWANPGKRGKTG

(19) United States
(12) Patent Application Publication
(10) Pub. No.: US 2007/0185020 A1
(43) Pub. Date: Aug. 9, 2007

(54) Methods and compositions for treating disorders of the extracellular matrix

(76) Inventors: Chai Zhonglin, Brunswick (AU); Mark Emmanuel Cooper, East Prahran (AU); Zemin Cao, Doncaster East (AU)

Correspondence Address: NIXON PEABODY LLP - PATENT GROUP, CLINTON SQUARE, P.O. BOX 31051, ROCHESTER, NY, 14603-1051, US

(21) Appl. No.: 10/562,778

(22) PCT Filed: Jun. 30, 2004
(86) PCT No.: PCT/AU04/00873

§ 371 Date: Jul. 27, 2006

(30) Foreign Application Priority Data
Jul. 1, 2003 (AU) 2003903363

(51) Int. Cl. A61K 38/17; A61K 48/00; G01N 33/567

(52) U.S. Cl. 514/12; 514/44; 435/7.2

(57) ABSTRACT

The present invention provides a method for altering the level of an extracellular matrix (ECM) protein produced by a cell, the method including modulating expression or activity of a cell division auto antigen (CDA). The Applicants have surprisingly found that a CDA is involved in a pathway that controls the level of ECM protein produced by mammalian cells. The invention has relevance to a number of ECM-related disorders such as renal fibrosis and atherosclerosis.

The present invention relates to the field of connective tissue biology. More specifically, the invention relates to the treatment of connective tissue disorders related to the extracellular matrix (ECM) such as fibrosis, and secondary diseases such as renal disease, diabetes and atherosclerosis.

### INTRODUCTION

The ECM is a gel-like material that normally has a structural role within connective tissue. The ECM is composed of ground substance and fibres. The ground substance is the amorphous substance that fills the interstitium and is composed of interstitial fluid, cell adhesion proteins and proteoglycans. Cells adhesion proteins allow the connective tissue cells to attach to matrix elements. The proteoglycans act to retain water thereby forming a semi-stiff hydrated gel.

The relative amounts and kinds of polysaccharides help determine the properties of the matrix. For example, the more polysaccharides the stiffer the ground substance is. The ground substance supports cells, binds them together and functions as a medium through which nutrients and other dissolved substances can diffuse between capillaries and cells.

Fibres in the matrix provide strength. Three types of fibres are found in the connective tissue matrix: collagen, elastic and reticular. Collagen fibres (white fibres) are extremely tough, providing high tensile strength, which is the ability to resist longitudinal stress. Since fresh collagen fibres have a glistening white appearance they are sometime called “white fibres”. Elastic fibres (yellow fibres) can be stretched to one and one-half times their length, but recoil to their initial length when released. They are found where greater elasticity is needed such as the lungs and the blood vessel walls. Fresh elastic fibres appear yellow and are also called yellow fibres. Reticular fibres are fine collagenous fibres. They form a delicate branching network supporting soft organs such as the liver and spleen.

While the ECM clearly fulfills an important structural role in the body, disorders of the ECM are common and serious. Fibrosis is a genera term for the generation of scar tissue in an animal, resulting from an overproduction of ECM. It has been estimated that 45 percent of the deaths in the United States are attributed to fibroproliferative disorders.

Fibrosis may result from diverse causes including trauma, surgery, infection, environmental pollutants, alcohol and other types of toxins. There are numerous examples of fibrosis, including the formation of scar tissue following a heart attack, which impairs the ability of the heart to pump. Diabetes frequently causes damage and scarring in the kidneys which leads to a progressive loss of kidney function. Even after surgery, scar tissue can form between internal organs causing contracture, pain, and in some cases, infertility.

Although fibrotic disorders can be acute or chronic, the disorders share a common characteristic of excessive collagen accumulation and an associated loss of function when normal tissue is replaced with scar tissue.

Acute fibrosis (usually with a sudden and severe onset and of short duration) occurs as a common response to various forms of trauma including accidental injuries, infections, surgery, burns, radiation and chemotherapy treatments. All tissues damaged by trauma are prone to scar and become fibrotic, particularly if the damage is repeated.

Chronic fibrosis can result from viral infection, diabetes, hypertension and other chronic conditions induce a progressive fibrosis which causes a continuous loss of tissue function. Most commonly affected are the liver, kidney and lung. Deep organ fibrosis is often extremely serious because the progressive loss of organ function leads to morbidity, hospitalization, dialysis, disability and even death.

Although disorders of the ECM are widely prevalent, debilitating and often life threatening, there is no effective treatment currently available. Given the adverse clinical effects of the many forms of ECM-related disorders there is a clear need for an effective treatment.

The Applicants have overcome or alleviated a problem of the prior art by discovering that a known cellular protein is involved in the pathophysiology of ECM disorders.

### SUMMARY OF THE INVENTION

In one aspect the present invention provides a method for altering the level of an extracellular matrix (ECM) protein produced by a cell, the method including modulating expression or activity of a cell division auto antigen (CDA). The Applicants have surprisingly found that a CDA is involved in a pathway that controls the level of ECM protein produced by mammalian cells. The invention has relevance to a number of ECM-related disorders such as renal fibrosis and atherosclerosis.

The CDA may be cell division autoantigen 1 (CDA1), or functional equivalent or derivative thereof.

In another aspect the present invention provides a method for treating or preventing a condition related to synthesis of an ECM protein, the method including modulating the expression and/or activity of a CDA.

The present invention also provides a non-human animal for use in studying disorders of the ECM having a cell capable of expressing CDA1 at an altered level.

Another aspect of the present invention provides a method of screening for an agent capable of modulating ECM synthesis, the method including the steps of
providing an animal or a cell capable of expressing CDA1,
exposing the animal or cell to the agent, and
determining the effect of the agent on CDA1 expression and/or activity.

The present invention further includes an agent identified by the screening methods described herein, as well as pharmaceutical compositions including the agent.

The present invention further includes a method for treating or preventing an ECM-related condition the method including administering to an animal in need thereof an effective amount of a pharmaceutical composition described herein.

Also provided by the present invention is a method of modulating CDA1 expression and/or activity in a cell, the method including exposing the cell to an agent capable of modulating the activity of a factor selected from the group including angiotensin II, TGFβ and connective tissue growth factor.

The present invention yet further provides a method of diagnosing a condition related to the synthesis of a ECM protein, the method including
obtaining a biological sample from the animal,
determining the level of CDA1 in the sample, and
comparing the level of CDA1 in the sample to a reference value
   wherein a positive diagnosis is made if the level of CDA1 in the sample is statistically significantly higher or lower than the reference value.

### DESCRIPTION OF THE FIGURES

FIG. 1 shows CDA1 expression in distal tubules and collecting ducts. The sections are counterstained with HE (nuclei are stained blue). Panel A; human tissue. Panel B; rat kidney tissue.

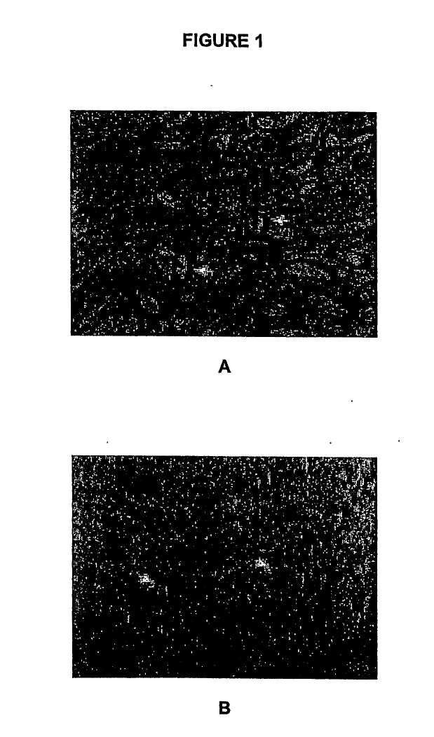

FIG. 2 shows increased CDA1 expression within the kidney in response to Ang II stimulation in vivo. Panel A; CDA1 expression control. Panel B; CDA1 expression after exposure to angiotensin II. Panel C; TGF-beta 1 expression after exposure to angiotensin II. Panel D; TGF-beta 2 expression after exposure to angiotensin II.

FIG. 3 shows increased CDA1 expression and fibrosis following renal mass reduction. Sections are counterstained with HE (nuclei stain blue). Panel A; CDA1 expression in subtotal nephrectomy.). Panel B; CDA1 expression in subtotal nephrectomy after exposure to valsartan.

FIG. 4 shows overexpression of CDA1 and production of extracellular matrix proteins in proximal tubule cells. Panel A; collagen IV staining in vehicle transfected NRK cells. Panel B; collagen IV staining in CDA1 transfected NRK cells. Panel C; fibronectin staining in vehicle transfected NRK cells. Panel D; fibrinectin staining in CDA1 transfected NRK cells.

FIG. 5 shows CDA1 expression in the kidney of a diabetic rat. Sections are counterstained with HE (nuclei stain blue). Panel A; CDA1 expression in control kidney. Panel B; CDA1 expression at 8 weeks; Panel C; CDA1 expression at 32 weeks.

FIG. 6 shows CDA1 expression in atherosclerotic plaques. CDA1 stains brown, with nuclei staining blue. Panel A; aorta of non-diabetic mouse. Panel B; aorta of diabetic (apoE) mouse.

FIG. 7 shows a DNA sequence that encodes human CDA1.

FIG. 8 shows a protein sequence for human CDA1.

### DETAILED DESCRIPTION OF THE INVENTION

In one aspect the present invention provides a method for altering the level of an extracellular matrix (ECM) protein produced by a cell, the method including modulating expression or activity of a cell division auto antigen (CDA). The Applicants have surprisingly found that a CDA is involved in a pathway that controls the level of ECM protein produced by mammalian cells.

The ECM is a complex structural entity surrounding and supporting cells that are found within mammalian tissues. The ECM is often referred to as the connective tissue. The ECM is composed of 3 major classes of biomolecules: structural proteins (collagen and elastin), specialized proteins (fibrillin, fibronectin, and laminin), and proteoglycans.

Accordingly, in a preferred form of the invention the ECM protein is selected from the group including collagen, elastin, fibrillin, fibronectin, laminin and proteoglycan. More preferably, the ECM protein is fibronectin or collagen IV.

As used herein, the term “modulating expression or activity of a CDA” means modifying or altering the expression and/or activity of a CDA compared with unmodified levels. Modulating expression may include inducing or increasing the expression and/or activity or reducing the expression and/or activity.

Modulation of CDA expression and/or activity in the cell may be achieved using antagonists, inhibitors, mimetics or derivatives of CDA. The terms “antagonist” or “inhibitor” as used herein refer to an agent which blocks or modulates a biological activity of CDA. Antagonists and inhibitors proteins, nucleic acids, carbohydrates, antibodies, or any other molecules or ligands. Proteins may include enzymes capable of degrading CDA thereby affecting the level of CDA in or around the cell. Other modulators of activity and/or expression of CDA include a range of rationally designed, synthetic inhibitors.

Modulation of CDA expression and/or activity may be achieved by direct or indirect methods. Modulation of expression and/or activity of CDA may be achieved using direct methods known to those skilled in the art and include, but are not limited to, knockout technology, antisense technology, triple helix technology, targeted mutation, gene therapy, regulation by agents acting on transcription. Indirect methods for modulating expression and/or activity of CDA include but are not limited to targeting upstream or downstream regulators such as cytokines.

The term “activity” relates to the function of a CDA in a cell, and includes the ability of CDA to bind to a chaperone molecule, or upstream or downstream effector molecules thereby activating or repressing upstream or downstream pathways which affect ECM protein production.

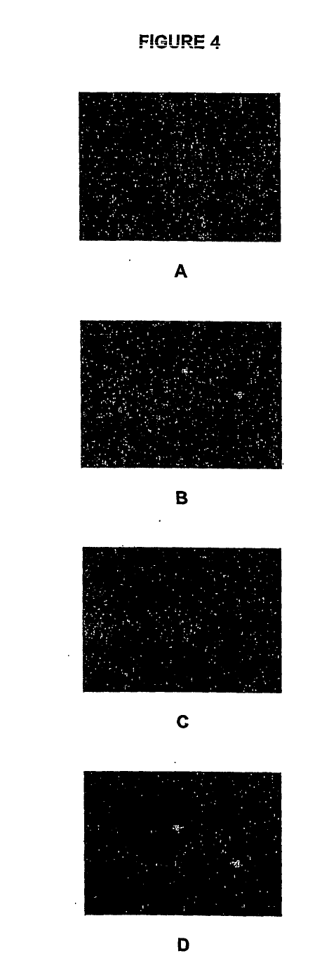

Preferably the cell originates from renal tissue or vascular tissue. There has been no previous report on CDA1 expression in the kidney. Applicants have shown that CDA1 is expressed in the kidney including distal tubules and collecting ducts (FIG. 4). CDA1 expression in the distal tubules and collecting ducts of normal rats demonstrated both cytoplasmic and nuclear patterns. CDA1 was rarely expressed in the glomeruli in the normal rat kidney.

More preferably the cell is selected from the group including a renal podocyte, renal proximal tubule cell, renal collecting duct cell, foam cell or macrophage.

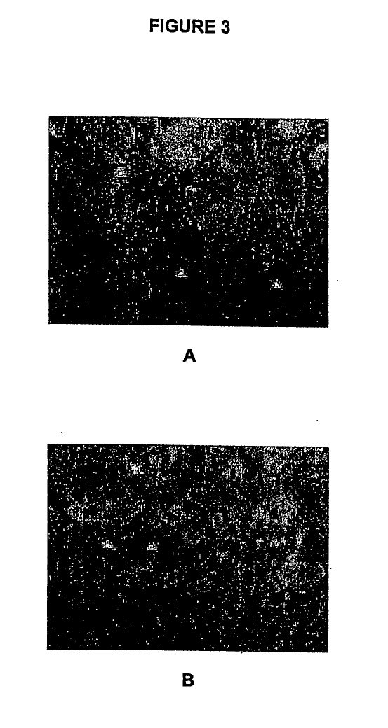

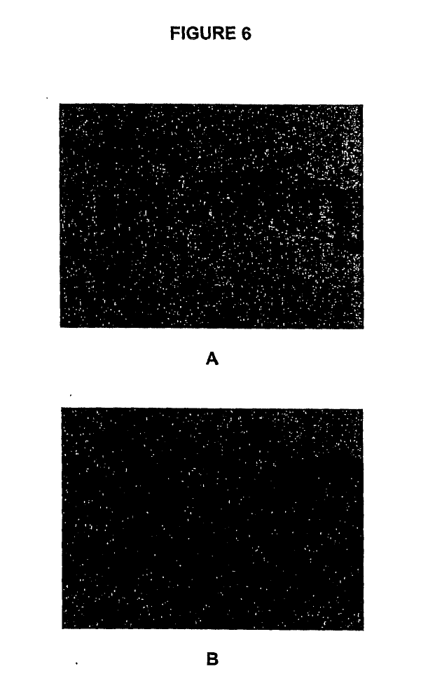

Co-localisation of CDA1 expression with fibrosis in the remnant kidney following subtotal nephrectomy (STNx) was also observed (FIG. 3). CDA1 expression appeared in the podocytes in the glomeruli following renal mass reduction (FIG. 6A) which was not seen in the normal kidney. Cytoplasmic and nuclear staining patterns were evident, particularly in the sclerotic glomeruli and at sites of tubulointerstitial fibrosis. Our preliminary data also indicate that therapeutic approaches which block the action of angiotensin II such as an AT1 receptor antagonist, valsartan, are associated with less cell injury and with attenuation of CDA1 expression (FIG. 3B).

Using the same CDA1 transfection techniques as described previously (Chai, et al (2001) SET-related Cell Division Autoantigen-1 (CDA1) Arrests Cell Growth. J. Biol. Chem. 276: 33665-33674, referred to hereinafter as “Chai, et al, 2001”), applicants have transfected CDA1 into the proximal tubular cell line, NRK-52E. Our preliminary studies have shown an increase in the production of matrix proteins including collagen IV (FIG. 4B) and fibronectin (FIG. 4D) in the CDA1 transfected cells when compared with cells transfected with vehicle alone (FIGS. 4A and 4C). This finding indicates that CDA1 may be directly linked to the production of extracellular matrix, and subsequently to the development of renal fibrosis.

In a preferred form of the invention the CDA is cell division autoantigen 1 (CDA 1), or a fragment, functional equivalent, analogue, mutant or variant thereof.

CDA1 is a novel nuclear protein recently identified using the serum of a patient with discoid lupus erythematosus, The Applicants have previously described the molecular cloning and structure of CDA1 in detail (see PCT/AU01/0148, published as WO 02/36768). In brief, the cDNA of CDA1 is 2808 base pairs and its open reading frame of 2079 base pairs encodes a predicted polypeptide of 693 amino acids named CDA1. CDA1 has a predicted molecular mass of 79,430 Daltons and a pl of 4.26.

CDA1 comprises an N-terminal proline-rich domain, a central basic domain, and a C-terminal bipartite acidic domain. The initial work by the Applicants have demonstrated that CDA1 has four putative nuclear localization signals and potential sites for phosphorylation by cAMP and cGMP-dependent kinases, protein kinase C, thymidine kinase, casein kinase 11, and cyclin-dependent kinases (CDKs). CDA1 is phosphorylated in HeLa cells and by cyclin D1/CDK4, cyclin A/CDK2, and cyclin B/CDK1 in vitro. Its basic and acidic domains contain regions homologous to almost the entire human leukemia-associated SET protein.

CDA1 expression is dynamic during the cell cycle and its level is directly related to the different stages of cell cycle and culture conditions. In serum starved cells (G₀ phase), CDA1 was expressed at a very low level. Addition of serum to culture media to stimulate the cell cycle (G1phase) was associated with an increase in CDA1 expression. When 2 mM thymidine was added to media to block cells at G1/S, CDA1 expression reached its peak. When cells were stopped at M phase by addition of 0.15 μg/ml colcemid, CDA1 expression was reduced but it was still higher than that detected at G₀ phase.

Other workers have subsequently identified CDA1 and demonstrated it as a TGF β1 target gene and named it Differentially Expressed Nucleolar TGF-β1 Target (DENTT). It was demonstrated that CDA1 expression was increased by TGF β1 in vitro. CDA1 (also known as DENTT) is highly expressed in the pituitary gland and moderately in the adrenals, brain, pancreatic islets, testis, and ovary suggesting a preponderance of this protein in endocrine organs.

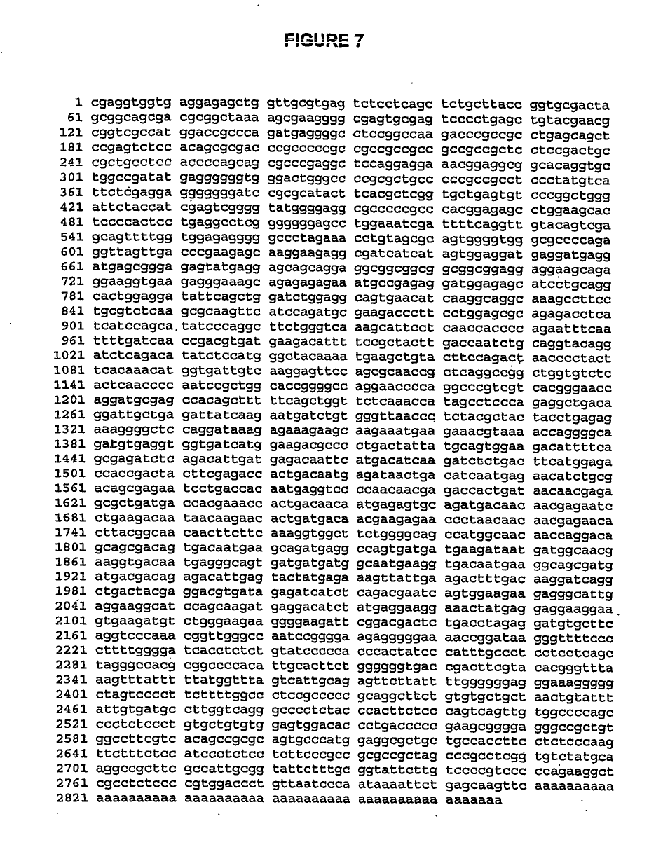

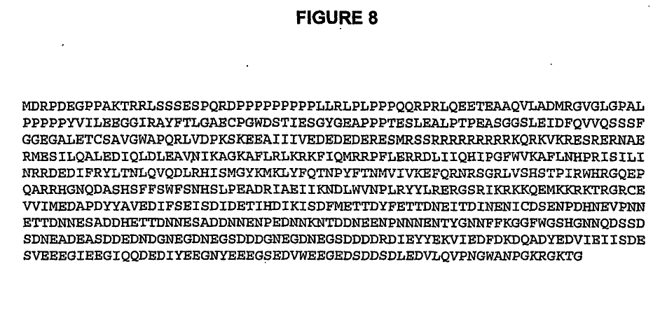

More preferably, the CDA1 is encoded by a nucleotide sequence according to FIG. 7, or a sequence that encodes a functional equivalent or derivative of CDA1. The CDA1 may nave an amino acid sequence according to FIG. 8 or functional equivalent or derivative thereof.

The term “functional equivalent or derivative” as used herein includes but is not limited to fragments, the fragments having the functional activity of CDA1 or homologues, analogues, mutants, variants and derivatives thereof. This includes homologues, analogues, mutants, variants and derivatives derived from natural, recombinant or synthetic sources including fusion proteins. Reference to “homologues” should be understood as a reference to CDA1 nucleic acid molecules or proteins derived from species other than the species being treated.

Derivatives include fragments, parts, portions, mutants, variants and mimetics from natural, synthetic or recombinant sources including fusion proteins Parts or fragments include, for example, active regions of CDA1. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutantic acid; asparagine and glutamine; serine and threonine; lysine and arginine; and phenylalanine and tyrosine. Additions to amino acid sequences include fusions with other peptides, polypeptides or proteins.

Chemical and functional equivalents of CDA1 nucleic acid or protein molecules should be understood to include molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Analogues contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences may similarly be derived from single or multiple nucleotide substitutions, deletions and/or additions including fusion with other nucleic acid molecules. The derivatives of the nucleic acid molecules of the present invention include oligonucleotides, PCR primers, antisense molecules, molecules suitable for use in cosuppression and fusion of nucleic acid molecules. Derivatives of nucleic acid sequences also include degenerate variants.

Examples of side chain modifications contemplated by the present invention include modifications of amino groups such as by reductive alkylation by reaction with an aldehyde followed by reduction with NaBH₄ amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodimmide activation via 0-acylisourea formation followed by subsequent derivitisation for example to a corresponding amide.

Sulphydryl groups may be modified by methods such as carboxymethylation with iodoacetic acid or iodoacetamide, performic acid oxidation to cysteic acid, formation of a mixed disulphides with other thiol compounds reaction with maleimide, maleic anhydride or other substituted maleimide, formation of mercurial derivatives using 4-chloromercuribenzoate, 4-chloromercuriphenylsulphonic acid, phenylmercury chloride, 2-chloromercuri-4-nitrophenol and other mercurials, carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids.

In another aspect the present invention provides a method for treating or preventing a condition related to synthesis of an ECM protein, the method including modulating the expression and/or activity of a CDA.

A condition related to synthesis of an ECM protein includes fibrosis as a result of trauma, and particularly injuries to spine and central nervous system. Also included are burns and other hypertrophic scars, cardiac scarring following heart attack, hemotherapeutic drug induced fibrosis, radiation induced fibrosis, lung fibrosis (Acute Respiratory Distress Syndrome) and surgical scarring.

The condition may also be major organ fibrosis including kidney disease (due to diabetes or hypertension for example), liver fibrosis (due to viral hepatitis or alcohol abuse for example), pulmonary fibrosis, cardiac fibrosis, macular degeneration, retinal and vitreal retinopathy.

The condition may also be a disorder such as systemic and local scleroderma, keloids and hypertrophic scars, atherosclerosis or restenosis

In a more preferred form of the invention the condition is renal fibrosis as a result of diabetes. Applicants have discovered increased expression of CDA1 in diabetic rat at 8 weeks (FIG. 6). Furthermore, renal CDA1 expression was further increased at 32 weeks after induction of diabetes. Increased CDA1 expression is found in the renal tubules and in the interstitium.

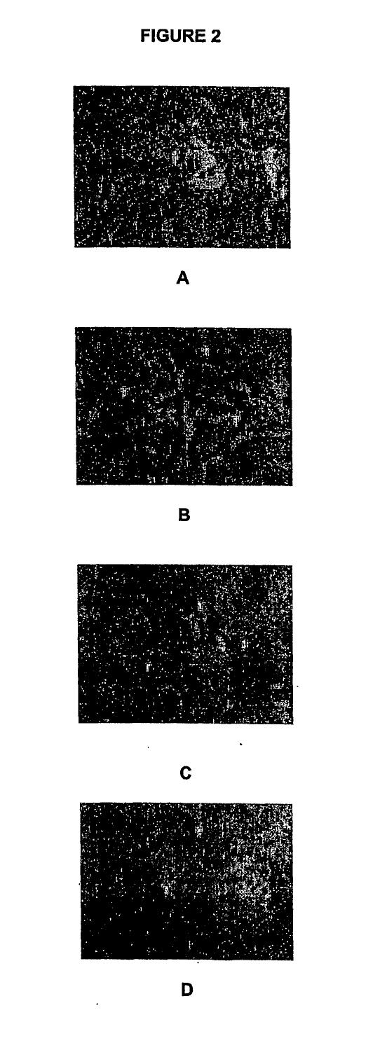

Of particular interest, there was de novo expression of CDA1 in podocytes from diabetic rats which was not seen in the kidneys from the normal rats (FIG. 2). Applicants have demonstrated that podocyte injury occurs in diabetic nephropathy and deficient expression of slit diaphragm proteins like nephrin may be partly responsible for the albuminuria seen in diabetes. Based on these data relating to CDA1 and known pathophysiological aspects of diabetic nephropathy, Applicants propose that this novel protein is an important link between functional and structural manifestations of diabetic nephropathy.

Without wishing to be limited by theory, it is proposed that hemodynamic and metabolic pathways which are activated in diabetes via angiotensin II, TGFβ, and CTGF, increase CDA1 expression thereby increasing the production of ECM proteins such as fibronectin and collagen IV.

In a further preferred embodiment of the invention the condition is atherosclerosis. Applicants have shown that CDA1 staining is increased in the atherosclerotic plaques in the aorta in diabetic Apo E knockout mice (FIG. 6), indicating that CDA1 may play a role in the development of atherosclerosis. Atherosclerosis is a common disorder of the arteries. Fat, cholesterol and other substances accumulate in the walls of arteries and form “atheromas” or plaques. Eventually, the fatty tissue can erode the wall of the artery, diminish the elasticity of the artery, and interfere with the blood flow.

Clots may form around the plaque deposits, further interfering with blood flow. When blood flow in the arteries to heart muscle becomes severely restricted, damage to the heart muscle is inevitable.

Risk factors include smoking, diabetes, obesity, high blood pressure, high blood cholesterol, a diet high in fats, and having a personal or family history of heart disease. Cerebrovascular disease, peripheral vascular disease, and kidney disease involving dialysis are also disorders that may also be associated with atherosclerosis. The present invention contemplates the treatment or prevention of atherosclerosis.

The present methods will also have use for treating conditions where an increase in ECM proteins is desirable, for example aneurysm and more particularly abdominal aortic aneurysm.

The present invention also provides a non-human animal for use in studying disorders of the ECM having a cell capable of expressing CDA1 at an altered level. The animal may be a “knock out” animal and express no CDA1 at all. The animal may express a CDA1 with decreased activity. It is contemplated that such an animal would be useful as a model for studying an ECM-related condition, or screening for agents useful in the treatment or prevention of any ECM-related condition.

Another aspect of the present invention provides a method of screening for an agent capable of modulating ECM synthesis, the method including the steps of
providing an animal or a cell capable of expressing CDA1,
exposing the animal or cell to the agent, and
determining the effect of the agent on CDA1 expression and/or activity.

The present invention further includes an agent identified by the screening methods described herein, as well as pharmaceutical compositions including the useful for treating an ECM related disease. Methods and carriers for the preparation of pharmaceutical and cosmetic compositions are well known in the art, as set out in textbooks such as Remington's Pharmaceutical Sciences, 18^{th} Edition, Mack Publishing Company, Easton, Pa., USA, the contents of which is incorporated herein.

The pharmaceutical compositions may be administered in a therapeutically or prophylactically effective amount for treating or preventing an ECM-related condition. The term “a therapeutically or prophylactically effective amount” as used herein means that amount necessary to at least partially attain the desired effect, or to delay the onset of, inhibit the progression of, or halt altogether, the onset or progression of an ECM-related condition. Such amounts may depend, of course, on the particular condition being treated, the severity of the condition and individual parameters, including age, physical condition, size, weight and other concurrent treatments. These factors are well known to those of ordinary skill in the art, and can be addressed with no more than routine experimentation. It is generally preferred that a minimum effective dose be determined according to sound medical or therapeutic judgement. It will be understood by those of ordinary skill in the art, however, that a higher dose may be administered for medical or other reasons. The skilled person will also be familiar with a range of administration routes and dosage regimes useful in the context of the present invention. No more than routine experimentation would be required to ascertain the optimal formulation, route of administration or dosage regime for a given clinical application.

The present invention further includes a method for treating or preventing an ECM-related condition the method including administering to an animal in need thereof an effective amount of a pharmaceutical composition described herein. The ECM-related condition may be any of those already described herein.

Also provided by the present invention is a method of modulating CDA1 expression and/or activity in a cell, the method including exposing the cell to an agent capable of modulating the activity of a factor selected from the group including angiotensin II, TGFβ and connective tissue growth factor.

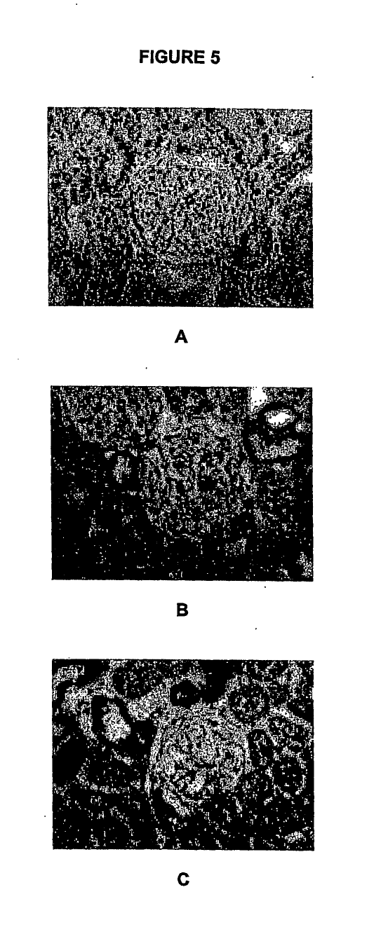

Applicants have shown increased CDA1 expression after angiotensin If infusion (FIG. 5B). Immunohistochemical staining for CDA1 showed increased levels of both cytoplasmic and nuclear staining in the proximal tubules. Increased CDA1 expression following Ang II infusion is prevented by treatment with valsartan, an AT1 receptor antagonist (data not shown).

The tubular pattern of increased CDA1 expression following angiotensin II infusion (FIG. 5B) is similar to that of TGF β (FIG. 2C) and TGF β2 (FIG. 2D). Furthermore, increased proximal tubular CDA1 expression was observed in the same sites as increased expression of p53 and bax, two important proapoptotic proteins (data not shown). The co-expression of p53 and bax is consistent with CDA1 being involved in inhibition of cell proliferation.

The present invention yet further provides a method of diagnosing a condition related to the synthesis of a ECM protein, the method including
obtaining a biological sample from the animal,
determining the level of CDA1 in the sample, and
comparing the level of CDA1 in the sample to a reference value
   wherein a positive diagnosis is made if the level of CDA1 in the sample is statistically significantly higher or lower than the reference value.

The method is contemplated to be useful in the diagnosis of any disease related to abnormalities of the ECF as described herein.

### EXAMPLES

### Example 1

### CDA1 Expression and Anq II: Time and Dose Relationship

Ang II was continuously infused into rats by subcutaneously inserted minipump for 3 days or 14 days at both pressor (60 ng/min) and non-pressor doses (6 ng/min) in eight week old male Sprague Dawley (SD) rats (Cao et al Z; The angiotensin type 2 receptor is expressed in adult rat kidney and promotes cellular proliferation and apoptosis. Kidney int. 2000; 58:2437-2451). The results are shown in FIG. 2.

### Example 2

### CDA1 Expression and Fibrosis in the Remnant Kidney

Subtotal nephrectomy (STNx) were performed by right nephrectomy, followed by infarction of approximately two-thirds of the left kidney with selective ligation of all but one extrarenal branch of the left renal artery (CIA40). Anaesthesia was achieved by intraperitoneal injection of pentobarbitone sodium (60 mg/kg). Sham operated rats were used as controls. After operation, the animals were sacrificed at 1, 2, 4, 8 and 12 week and tissue collected for assessment of CDA1 expression and fibrotic growth factors as well as matrix proteins. The results are shown in FIG. 3.

### Example 3

### Effects of CDA1 Overexpression on Expression of Collagens, Osteopontin, Fibronectin in the Presence or Absence of Ang II, TGF β and CTGF Stimulation

Over expression of CDA1 was achieved by CDA1 transfection in a cultured normal rat kidney epithelial cell line. Overexpression of CDA1 was achieved by CDA1 transfection in a cultured normal rat kidney epithelial cell line (NRK52E). A construct expressing Myc-tagged CDA1, namely cDNA3-2M-CDA1 and pcDNA3-2M vector control DNA were used to transfect NRK52E cells by electroporation as described for transfecting HeLa cells previously (Chai, et al, 2001)

The transfected cells were plated on coverslips and cultured in DMEM without foetal bovine serum for three days. Fibronectin and other matrix proteins were assessed by immunohistochemistry. Gene expression of TGF beta, collagen I, and fibronectin may be assessed by real time RT-PCR or immunochemistry as described in the following citations: Cao et al Blockade of the renin angiotensin and endothelin systems on progressive renal injury. Hypertension 2000; 36:561-568; Twigg et al Renal connective tissue growth factor induction in experimental diabetes is prevented by aminoguanidine. Endocrinology. 2002;143:4909-4913

Extracellular matrix proteins were assessed in the presence or absence of CDA1 or vehicle transfected cells by immunohistochemistry. The results are shown in FIG. 4.

Diabetes was induced in Sprague Dawley (SD) rats aged 8 weeks by intravenous injection of streptozotocin (STZ) (Allen et al Role of angiotensin II and bradykinin in experimental diabetic nephropathy—functional and structural studies. Diabetes.1997; 46:1612-1618). Animals were sacrificed after 8 and 32 weeks of diabetes, and kidneys removed for subsequent immunohistochemistry for CDA1. The results are shown in FIG. 5

STZ diabetes was induced in apo E knockout mice (6 daily injections of STZ), and after 20 weeks of diabetes, aortae were removed for immunohistochemistry. The results are shown in FIG. 6.

### Example 4

### Method for CDA 1 Immunohistochemistry

CDA1 staining was performed using a rabbit antihuman CDA1 serum (Chai et al, 2001) in paraffin embedded sections. In brief, following dewaxing, paraffin embedded sections were treated in a microwave oven at low power for 10 minutes in 10 mmol/L sodium citrate buffer (pH 6.0). Endogenous peroxidase was inactivated using 3% hydrogen peroxide (H₂O₂) in methanol for 20 minutes. The sections were blocked with protein block agent for 20 minutes. The sections were incubated with rabbit antihuman CDA1 serum for 2 hour at room temperature. Biotinylated goat anti-rabbit immunogloblulin (DAKO A/S) was used as a second antibody, followed by avidin biotin horseradish peroxidase complex (ABC Elite kit, Victor Laboratories, Burlingame, Calif.). Detection was accomplished by reaction with 3,3′-diaminobenzidine tetrahydrochloride (DAB, Sigma Chemical Co., St Louis, Mo.) (Cao Z et al. The angiotensin type 2 receptor is expressed in adult rat kidney and promotes cellular proliferation and apoptosis. Kidney Int. 2000; 58:2437-2451.

Finally it is to be understood that various other modifications and/or alterations may be made without departing from the spirit of the present invention as outlined herein.

1. A method for altering the level of an extracellular matrix (ECM) protein produced by a cell, the method including modulating expression or activity of a cell division auto antigen (CDA).

2. A method according to claim 1 wherein the ECM protein is selected from the group consisting of collagen, elastin, fibrillin, fibronectin, laminin and proteoglycan.

3. A method according to claim 1 wherein the ECM protein is fibronectin or collagen IV.

4. A method according to claim 1 wherein the cell originates from renal tissue or vascular tissue.

5. A method according to claim 1 wherein the cell is selected from the group consisting of a renal podocyte, a renal proximal tubule cell, a renal collecting duct cell, a foam cell and a macrophage cell.

6. A method according to claim 1 wherein the CDA comprises an N-terminal proline-rich domain, a central basic domain, and a C-terminal bipartite acidic domain.

7. A method according to claim 1 wherein the CDA is cell division autoantigen 1 (CDA 1), or a fragment, functional equivalent, analogue, mutant or variant thereof.

8. A method according to claim 7 wherein the CDA1 is encoded by a nucleotide sequence according to SEQ ID NO:1.

9. A method according to claim 7 wherein the CDA1 has an amino acid sequence according to SEQ ID NO:2 or functional equivalent or derivative thereof.

10. A method for treating or preventing a condition related to synthesis of an ECM protein, the method including modulating the expression and/or activity of a CDA.

11. A method according to claim 10 wherein the condition is fibrosis.

12. A method according to claim 11 wherein the fibrosis is due to a burn, a heart attack, treatment with a chemotherapeutic drug, exposure to radiation, or surgery.

13. A method according to claim 11 wherein the fibrosis is major organ fibrosis.

14. A method according to claim 13 wherein the major organ is selected from the group consisting of kidney, liver, heart and eye.

15. A method according to claim 13 wherein the major organ fibrosis is due to a condition selected from the group consisting of diabetes, hypertension, viral hepatitis, alcohol abuse, macular degeneration, retinal retinopathy and vitreal retinopathy.

16. A method according to claim 11 wherein the condition is renal fibrosis as a result of diabetes.

17. A method according to claim 10 wherein the condition is selected from the group including systemic and local scleroderma, keloids, hypertrophic scars, atherosclerosis and restenosis.

18. A method according to claim 17 wherein the condition is atherosclerosis.

19. A method according to claim 10 wherein the condition is aneurysm.

20. A method according to claim 19 wherein the aneurysm is abdominal aortic aneurysm.

21. A method according to claim 10 wherein the CDA is CDA1.

22. A non-human animal for use in studying disorders of the ECM, the animal having a cell capable of expressing a CDA at an altered level.

23. A non-human animal according to claim 22 wherein the CDA is CDA1.

24. A method of screening for an agent capable of modulating ECM synthesis, the method including the steps of
providing an animal or a cell capable of expressing a CDA,
exposing the animal or cell to the agent, and
determining the effect of the agent on the CDA expression and/or activity.

25. A method according to claim 24 wherein the CDA is CDA1.

26. An agent identified by the method according to claim 24.

27. A pharmaceutical composition including an agent according to claim 26.

28. A method for treating or preventing a condition related to an ECM protein, the method including administering to an animal in need thereof an effective amount of a pharmaceutical composition according to claim 27.

29. A method of modulating CDA expression and/or activity in a cell, the method including exposing the cell to an agent capable of modulating the expression and/or activity of a factor selected from the group consisting of angiotensin II, TGFβ and connective tissue growth factor.

30. A method according to claim 29 wherein the CDA is CDA1.

31. A method of diagnosing a condition related to the synthesis of a ECM protein in an animal, the method including
obtaining a biological sample from the animal,
determining the level of CDA in the sample, and
comparing the level of CDA in the sample to a reference value
wherein a positive diagnosis is made if the level of CDA in the sample is statistically significantly higher or lower than the reference value.

32. A method according to claim 31 wherein the CDA is CDA1.
